(19) United States
(12) Patent Application Publication
(10) Pub. No.: US 2006/0173487 A1
(43) Pub. Date: Aug. 3, 2006

(54) Angioplasty cutting device and method for treating a stenotic lesion in a body vessel

(75) Inventors: Renan Uflacker, Mount Pleasant, SC (US); Kian Olsen, Bloomington, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 11/325,977

(22) Filed: Jan. 5, 2006

Related U.S. Application Data
(60) Provisional application No. 60/641,801, filed on Jan. 5, 2005.

(51) Int. Cl. A61M 29/00

(52) U.S. Cl. 606/198

(57) ABSTRACT

An angioplasty cutting device for balloon angioplasty of a stenotic lesion in a body vessel. The device comprises a distal ring and a proximal ring. The device further comprises at least one strut attached to the distal ring and proximally extending to the proximal ring. The strut is configured to be disposed at the stenotic lesion to engage the stenotic lesion for dilatation of the body vessel during angioplasty. The strut extending from the proximal ring a predetermined length for delivery and retrieval of the device.

CROSS-REFERENCE TO RELATED APPLICATIONS

This application claims the benefit of U.S. Provisional Application Ser. No. 60/641,801, filed on Jan. 5, 2005, entitled “ANGIOPLASTY CUTTING DEVICE AND METHOD FOR TREATING A STENOTIC LESION IN A BODY VESSEL,” the entire contents of which are incorporated herein by reference.

### BACKGROUND OF THE INVENTION

The present invention relates to medical devices. More particularly, the present invention relates to angioplasty cutting devices and methods for treating a stenotic lesion in a body vessel.

Vascular diseases, such as coronary artery disease, are common diseases. Such diseases are caused by stenotic lesions narrowing in a body vessel within the vasculature. Generally, carotid artery stenosis is the narrowing of the carotid arteries, the main arteries in the neck that supply blood to the brain. Carotid artery stenosis (also called carotid artery disease) is a relatively high risk factor for ischemic stroke. The narrowing is usually caused by plaque build-up in the carotid artery. Plaque forms when cholesterol, fat and other substances form in the inner lining of an artery. This formation is called atherosclerosis.

Currently, depending on the degree of stenosis and the patient's overall condition, carotid artery stenosis can usually be treated with surgery. The procedure is (with its inherent risks) called carotid endarterectomy, which removes the plaque from the arterial walls. Carotid endarterectomy has proved to benefit patients with arteries stenosed by about 70% or more. For people with arteries narrowed less than 50%, an anti-clotting agent may be prescribed to reduce the risk of ischemic stroke.

Carotid angioplasty is another treatment for carotid artery stenosis. This treatment uses balloons and/or stents to open a narrowed artery. Carotid angioplasty is a procedure that can be performed via a standard percutaneous transfemoral approach with the patient anesthetized using light intravenous sedation. At the stenosis area, an angioplasty balloon is delivered to predilate the stenosis in preparation for stent placement. The balloon is then removed and exchanged via catheter for a stent delivery device. Once in position, a stent is deployed across the stenotic area. If needed, an additional balloon can be placed inside the deployed stent for post-dilation to make sure the struts of the of the stent are pressed firmly against the inner surface of the vessel wall.

However, an ongoing problem with angioplasty is that the arterial blockage may return, usually within 6 months. It is thought that the mechanism of this phenomenon, called “restenosis,” is not the progression of the arterial disease, but rather the body's immune system response to the angioplasty. At this point, a repeat procedure may need to be performed.

Thus, there is a need to provide a way for decreasing the likelihood of restenosis without the inherent risks of surgery.

### BRIEF SUMMARY OF THE INVENTION

The present invention generally provides a cutting assembly, a cutting device, and method for treating a stenotic lesion of a body vessel, decreasing the likelihood of restenosis without the inherent risks of surgery. Embodiments of the present invention provide a simple, efficient and cost effective way of treating atherosclerosis and stenosis of a body vessel. For example, the cutting device of the present invention provides an effective, efficient way of breaking plaque of a stenotic lesion while using various sizes of angioplasty balloons.

One embodiment of the present invention is an angioplasty cutting device for balloon angioplasty of a stenotic lesion in a body vessel. The device comprises a distal ring configured to be disposed at the distal end of the stenotic lesion relative to the device. The device further comprises at least one strut attached to the distal ring and proximally extending therefrom. The at least one strut is configure to be disposed at the stenotic lesion to engage the stenotic lesion for dilatation of the body vessel during angioplasty. The device further comprises a proximal ring configured to be disposed at the proximal end of the stenotic lesion relative to the device. The at least one strut is attached to the proximal ring and extends therefrom a predetermined length for delivery and retrieval of the device.

In another embodiment, the present invention provides an atherosclerosis cutting device coaxially adaptable about an expandable balloon for angioplasty of a stenotic lesion in a body vessel. The device comprises a plurality of struts defining a cutting body wherein each strut has a first portion and a second portion. Each first portion is attached to the distal ring and extends longitudinally therefrom. The cutting body is radially expandable with the balloon to engage the stenotic lesion for dilatation of the body vessel during angioplasty. The device further comprises a proximal ring configured to be disposed adjacent the proximal end of the balloon relative to the device. Each second portion is attached to the proximal ring and one of the struts extends therefrom a predetermined length for delivery and retrieval of the device.

Yet another embodiment of the present invention is an angioplasty cutting apparatus for treatment of a stenotic lesion in a body vessel. The apparatus comprises a balloon catheter having a tubular body and an expandable balloon attached to an in fluid communication with the tubular body for angioplasty at the stenotic lesion. The expandable balloon has distal and proximal portions. The apparatus further includes an angioplasty cutting device coaxially adaptable about the expandable balloon for angioplasty of the stenotic lesion in the body vessel. The device generally comprises a distal ring, a cutting body, and a proximal ring. The distal ring is configured to be disposed adjacent the distal end of the balloon relative to the device. The cutting body includes a plurality of struts, wherein each strut has a first portion and a second portion. Each first portion is attached to the distal ring and extends longitudinally therefrom. The cutting body is radially expandable with the balloon to engage the stenotic lesion for dilatation of the body vessel during angioplasty. The proximal ring is configured to be disposed adjacent the proximal end of the balloon relative to the device. Each second portion is attached to the proximal ring. One of the struts extends therefrom a predetermined length for delivery and retrieval of the device.

Further objects, features, and advantages of the present invention will become apparent from consideration of the following description and the appended claims when taken in connection with the accompanying drawings.

### BRIEF DESCRIPTION OF THE DRAWINGS

FIG. 1 is an environmental view of an atherosclerosis cutting device for balloon angioplasty of a stenotic lesion in a body vessel in accordance with one embodiment of the present invention;

FIG. 2 is another environmental view of the cutting device for balloon angioplasty of the stenotic lesion in the body vessel;

FIG. 3 is a side view of the cutting device depicted in FIGS. 1 and 2;

FIG. 4 is a cross-sectional view of the cutting device of FIG. 2 taken along line 4-4;

FIG. 5 is an end view of the cutting device of FIG. 2;

FIG. 6a is a cross-sectional view of the cutting device in a collapsed state;

FIGS. 6b-6d are cross-sectional views of the cutting device in transition states during inflation of the expandable balloon;

FIG. 6e is a cross-sectional view of the cutting device in an expanded state;

FIG. 7a is an exploded view of an assembly implementing the atherosclerosis cutting device in accordance with one embodiment of the present invention;

FIG. 7b is a side view of the assembly of FIG. 7a;

FIG. 8 is a flowchart depicting one method of treating a stenotic lesion in a body vessel using the atherosclerosis cutting device;

FIG. 9 is an environmental view of an atherosclerosis cutting device for balloon angioplasty of a stenotic lesion in a body vessel in accordance with another embodiment of the present invention;

FIG. 10 is another environmental view of the cutting device of FIG. 9 for balloon angioplasty of the stenotic lesion in the body vessel; and

FIG. 11 is a side view of the angioplasty cutting device depicted in FIGS. 9 and 10.

### DETAILED DESCRIPTION OF THE INVENTION

The present invention generally provides a cutting assembly, a cutting device, and method for treating a stenotic lesion of a body vessel. Embodiments of the present invention provide a more simple, efficient and cost effective way of treating atherosclerosis and stenosis of a body vessel. For example, the cutting device includes a cutting body disposed about one of a number of various-sized expandable balloons of a balloon catheter for angioplasty. The cutting body has one or a plurality of struts which expand as the expandable balloon is inflated. Each strut defines a focal point or a radial plane of fracture on the stenotic lesion whereat lacerations on the stenotic lesion are formed. Upon contact with the stenotic lesion, the struts break the plaque in a relatively organized fashion, lessening the likelihood of restenosis of the body vessel.

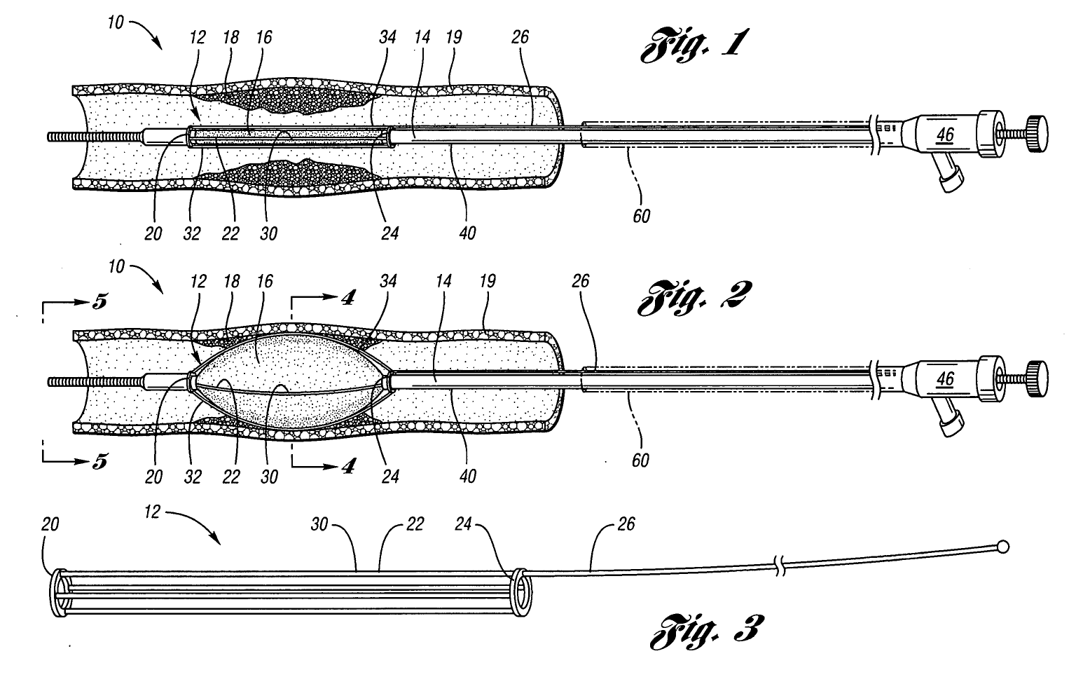

FIG. 1 illustrates an angioplasty cutting assembly or apparatus 10 having an expandable balloon 16 and implementing an atherosclerosis or angioplasty cutting device 12 in accordance with one embodiment of the present invention. As depicted in FIGS. 1 and 2, the cutting assembly 10 includes a balloon catheter 14 about which the device 12 is disposed. As shown, the balloon catheter 14 comprises an expandable balloon 16 for angioplasty treatment of a stenotic lesion 18 of a body vessel 19. The balloon catheter 14 is configured to be cooperable with the device 12 during the procedure. As shown, the device 12 is disposable about the expandable balloon 16 of the assembly 10. As the balloon is inflated, the device 12 expands to engage the stenotic lesion 18 of the body vessel 19.

FIG. 1 further depicts the assembly 10 in a deflated or an unexpanded state or condition that the device 12 takes on during delivery and retrieval thereof. FIG. 2 shows the assembly 10 in an inflated or an expanded state that the device 12 takes on during angioplasty. The expandable balloon 16 of the assembly 10 may be inflated and deflated by any suitable means, e.g., by introducing saline into the expandable balloon 16 as known in the art.

FIGS. 1-3 generally illustrate the device 12 comprising a distal ring 20, a cutting body 22 extending from the distal ring 20, a proximal ring 24 to which the cutting body 22 extends, and a retrieval member 26 proximally extending from the proximal ring 24. The cutting body 22 defines at least one radial plane of fracture A in the body vessel 19 during angioplasty.

As shown, the distal ring 20 is preferably a ring member located at the distal portion of the cutting device 12. In this embodiment, the distal ring 20 is configured to be disposed about and adjacent the distal end 21 of the expandable balloon 16 relative to the device 12. The distal ring 20 may be made of any suitable material. Such materials may include superelastic material (e.g. Nitinol), metals (e.g., stainless steel), high density polymeric material (e.g., high density polyethylene or polypropylene).

In this embodiment, the cutting body 22 includes a plurality of struts or wires 30 attached to the distal ring 20 and extending to the proximal ring 24. However, it is to be noted that the cutting body 22 may include merely one strut extending from the distal ring 20 to the proximal ring 24. Each strut is preferably attached to the distal ring 20 and extends proximally longitudinally therefrom. Preferably, each strut has a first portion 32 and a second portion 34. In this embodiment, the first portion 32 is a distal portion, and the second portion 34 is a proximal portion relative to the device 12. The cutting body 22 is radially expandable with the balloon to engage the stenotic lesion 18 for dilatation of the body vessel 19 during angioplasty. As shown, each strut is configured to be placed at the stenotic lesion 18 and to extend longitudinally along the length of the stenotic lesion 18.

Preferably, each strut defines a focal point or a radial plane of fracture A whereat lacerations to the stenotic lesion 18 are formed during angioplasty. That is, the struts 30 cut the plaque of the lesion at focal points to provide the radial planes of fracture A to the lesion, thereby dilating the body vessel 19. During angioplasty, each strut 30 of the cutting body 22 expands along its respective radial plane of fracture A to engage the stenotic lesion 18 in the body vessel 19. Upon contact with the lesion, the struts 30 break the plaque in a relatively organized fashion. It has been found that, as the expandable balloon pushes the lesion radially outwardly, the struts cut and allow the plaque to be folded for further dilatation of the body vessel. Furthermore, trauma to the lesion caused by the struts 30 result in relatively organized lacerations that minimize or lessen the likelihood of restenosis of the body vessel. Thus, the lacerations formed on the lesion allow for a relatively more effective treatment of stenosis.

The struts 30 may be made of a rigid material, a superelastic material or a shape memory material. For example, the struts 30 may be made of stainless steel, Nitinol, or a polymeric material (e.g., high density polyethylene or polypropylene). Preferably, each of the struts 30 may have a diameter of between about 0.014 inch and 0.018 inch.

Preferably, each strut is attached to the distal ring 20 such that the device 12 may be radially placed about the expandable balloon 16. Each strut is attached to the distal ring 20 by bonding. This may be accomplished by sonic bonding, thermal bonding, or adhesive bonding. As shown, the struts 30 proximally extend from the distal ring 20 to attach to the proximal ring 24. The proximal ring 24 is configured to be disposed about and adjacent the proximal end 25 of the expandable balloon 16 relative to the device 12. The proximal ring 24 may be made of any suitable material. Such materials may include superelastic material (e.g. Nitinol), metals (e.g., stainless steel), high density polymeric material (e.g., high density polyethylene or polypropylene). In this embodiment, each second portion 34 of each respective strut is attached to the proximal ring 24. Preferably, each strut is attached to the proximal ring 24 such that the device 12 may be radially disposed about the expandable balloon 16. Each strut may be attached to the proximal ring 24 by bonding, e.g., sonic bonding, thermal bonding, or adhesive bonding.

At least one of the struts 30 extends past the proximal ring 24 a predetermined length for delivery and retrieval of the device 12. It is to be noted that one or more struts 30 may extend through the proximal ring 24. Alternatively, a retrieval wire or strut may be attached to the proximal ring 24 and extend proximally therefrom a predetermined length for delivery and retrieval of the device 12. Also, it is to be understood that each of the struts 30 may be integrally connected to the distal ring 20 or the proximal ring 24. This may be accomplished by any suitable means such as by molding or casting the device 12 to provide a single member device 12.

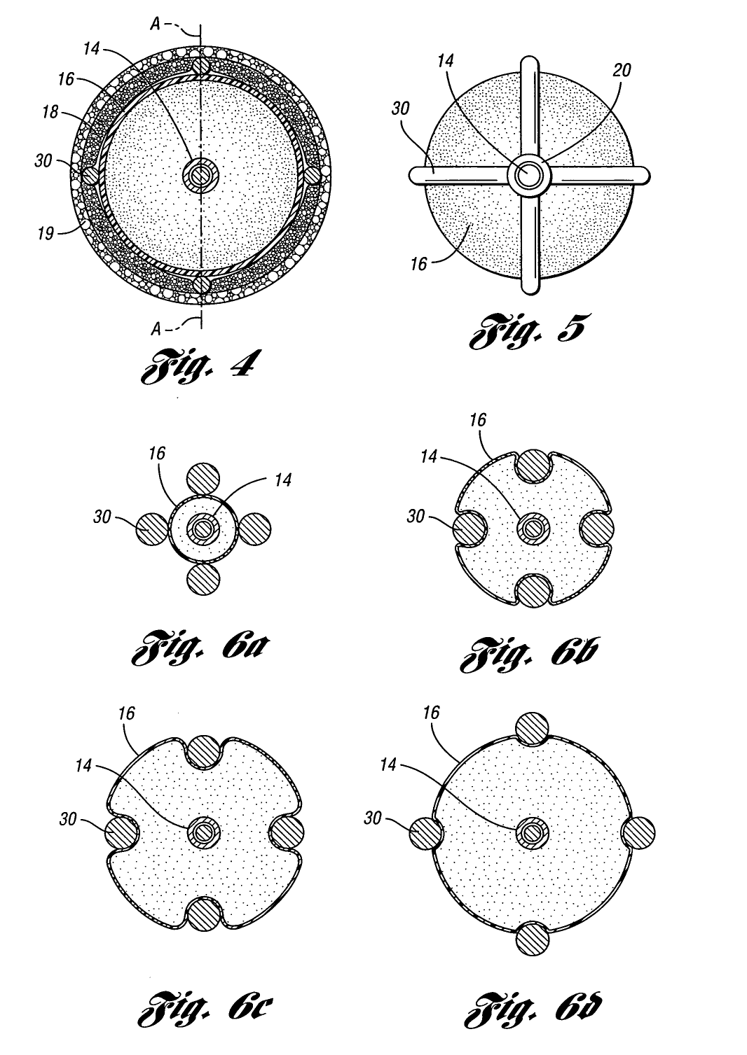

The condition of the device 12 is dictated by the condition of the expandable balloon 16 of the assembly 10. FIGS. 4 and 5 depict cross-sectional and end views of the device 12 taken along lines 4-4 and 5-5 of FIG. 2, respectively. As shown, the expansion of the struts 30 of the vehicle are dictated by the inflation of the angioplasty balloon such that each strut expands along its respective radial plane of fracture to contact and fracture the stenotic lesion 18, thereby lessening the likelihood of restenosis.

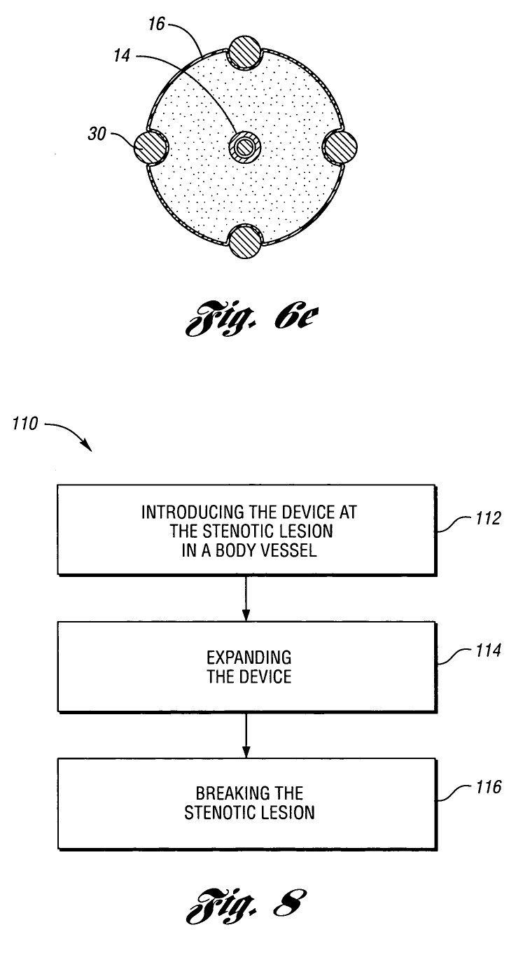

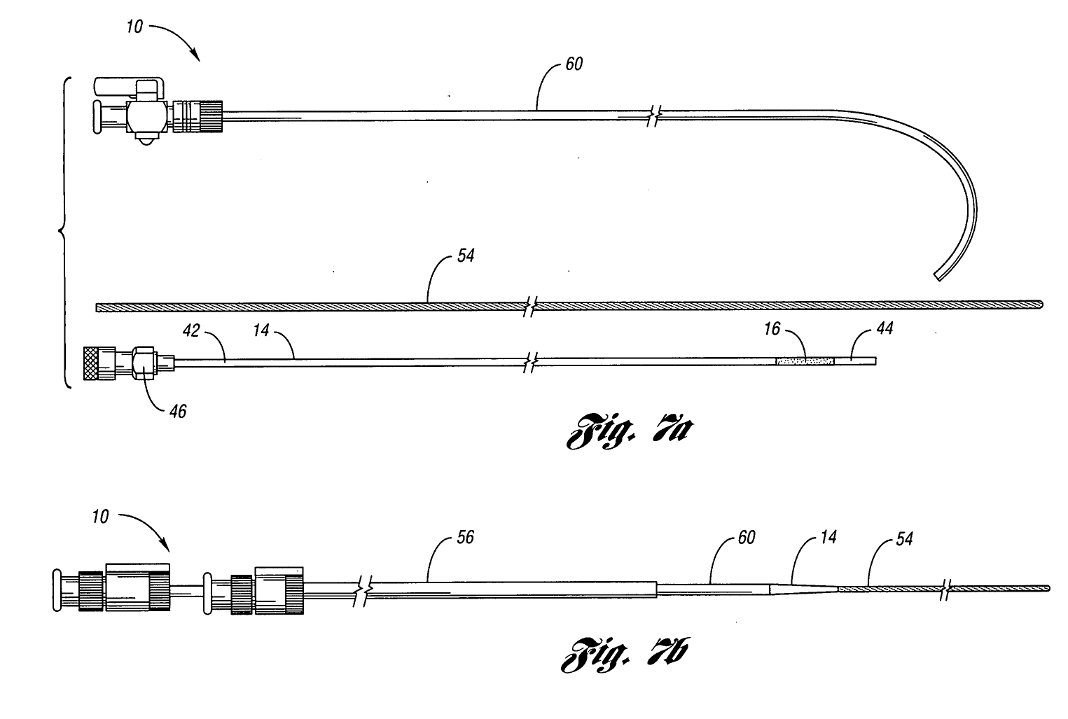

FIGS. 6a-6d depict states that the device 12 takes on during a stenotic procedure as the expandable balloon 16 is inflated to engage the struts 30 with the stenotic lesion 18. FIG. 6a illustrates the device 12 in a collapsed state. In the collapsed state, the device 12 and assembly 10 may be delivered to and retrieved from a stenotic lesion 18. In this embodiment, the outer diameter of the expandable balloon is about 0.5 to 3 millimeters (mm).

FIGS. 6b-6d illustrate the device 12 in transition states during inflation of the expandable balloon 16. During the transition states, the device 12 may begin contacting the stenotic lesion 18. In this embodiment, in FIG. 6b, the outer diameter of the expandable balloon is about 3 to 6 mm. In FIGS. 6c and 6d, the outer diameter of the expandable balloon is about 4 to 8 mm.

FIG. 6e depicts the device 12 in an expanded state as the balloon inflation is completed. In the expanded state, the struts 30 of the device 12 are preferably in contact or relatively near contact with the vessel wall and have fractured the stenotic lesion 18. The organized fracturing and trauma to the stenotic lesion 18 provides a lessened likelihood of restenosis of the body vessel. In this embodiment, the outer diameter of the expandable balloon is about 5 to 10 mm.

FIGS. 7a-7b depict a cutting assembly 10 which implements the cutting device 12 for treating a stenotic lesion 18 of a body vessel in accordance with one embodiment of the present invention. As shown, the assembly 10 includes the balloon catheter 14 having a tubular body 40 portion and an expandable balloon 16 disposed thereon. The expandable balloon 16 is preferably attached to and in fluid communication with the tubular body 40 for angioplasty at the stenotic lesion 18. The device 12 is configured to be disposed about the expandable balloon 16 for deployment at the stenotic lesion 18. The device 12 is preferably placed about the angioplasty balloon of the angioplasty catheter prior to insertion into the vasculature.

Generally, the balloon catheter 14 has a proximal end 42, a distal end 44, and a plastic adapter or hub 46 to receive assembly 10 to be advanced therethrough. The hub 46 is in fluid communication with the balloon for fluid to be passed therethrough for inflation and deflation of the balloon during angioplasty. In one embodiment, the balloon catheter 14 may include an outer lumen 50 and an inner lumen 52. The outer lumen 50 is preferably in fluid communication with the expandable balloon 16 for inflating and deflating the balloon. The inner lumen 52 is formed therethrough for percutaneous guidance through the body vessel. The balloon catheter 14 is preferably made of a soft, flexible material such as a silicone or any other suitable material. In this embodiment, the inside diameter of the balloon catheter 14 may range between 0.014 and 0.027 inch.

The size of the expandable balloon 16 may also vary. For example, the balloon size may range between about 2 and 10 millimeters in diameter. The expandable balloon 16 has distal and proximal portions. The expandable balloon 16 may be made of any suitable material such as low density polymer material such as polyvinyl chloride.

The assembly 10 further includes a wire guide 54 which via an introducer sheath 56 (discussed in greater detail below) is percutaneously inserted to provide a path for the balloon catheter 14 within the vasculature of a patient. The balloon catheter 14 is configured to be disposed about the wire guide 54 for percutaneous guidance through the vasculature. The size of the wire guide 54 is based on the inside diameter of the introducer sheath 56.

As mentioned above, the assembly 10 further includes a polytetrafluoroethylene (PTFE) introducer sheath 56 for percutaneously introducing the wire guide 54 and the balloon catheter 14 in vasculature. Of course, any other suitable material may be used without falling beyond the scope or spirit of the present invention. The introducer sheath 56 is percutaneously inserted into the vasculature of the patient. The sheath may have a size of about 4-French to 8-French and allows the balloon catheter 14 to be inserted therethrough to the deployment location in the body vessel. In one embodiment, the sheath receives the balloon catheter 14 and the device 12, and provides stability thereto at the deployment location.

The assembly 10 may further include an outer catheter 60 disposed co-axially about the balloon catheter 14 within the introducer sheath 56. As shown, the outer catheter 60 is preferably configured to house the balloon catheter 14 and the device 12 during delivery and retrieval thereof to and from the stenotic lesion 18. The outer catheter 60 is preferably advanced with the balloon catheter 14 and the device 12 to the deployment location. When the distal end 21 of the expandable balloon 16 of the balloon catheter 14 is placed across the stenotic lesion 18 in the body vessel, the expandable balloon 16 may then be inflated preferably with saline. For deployment of the expandable balloon 16 and the cutting device 12, the outer catheter 60 is then retracted to expose the device 12 and angioplasty balloon at the stenotic lesion 18. The angioplasty balloon is inflated, and both the device 12 and balloon expands to break plaque of the stenotic lesion 18.

It is to be understood that the assembly 10 described above is merely one example of an assembly 10 that may be used to deploy the capturing device 12 in a body vessel. Of course, other apparatus, assemblies, and systems may be used to deploy any embodiment of the capturing device 12 without falling beyond the scope or spirit of the present invention.

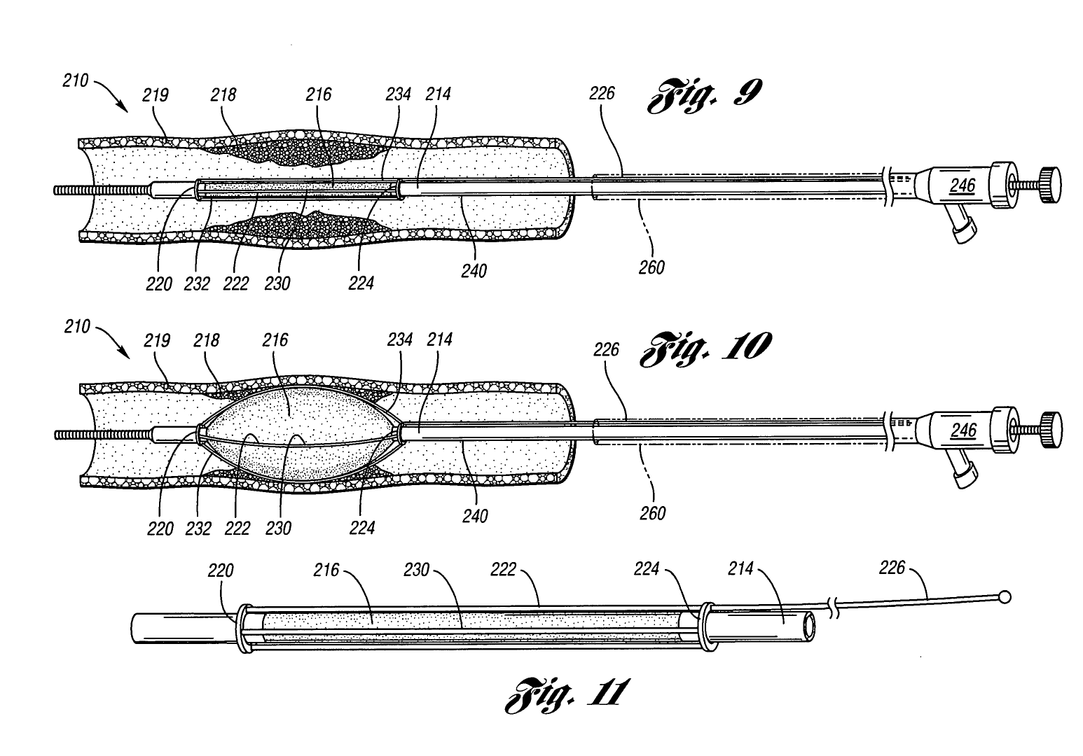

FIG. 8 illustrates a flow chart depicting one method 110 for treating a stenotic lesion 18 in a body vessel, implementing the assembly 10 mentioned above. The method 110 comprises percutaneously introducing an expandable balloon 16 at a stenotic lesion 18 in the body vessel in box 112. The method 110 further comprises disposing the cutting device 12 coaxially about the expandable balloon 16 for angioplasty of the stenotic lesion 18 in the body vessel. The method further includes passing saline through the balloon catheter 14 to the expandable balloon 16 to contact the balloon and the device 12 on the stenotic lesion 18. The method 110 further includes inflating the expandable balloon 16 and expanding in box 114 the cutting device 12 for contact with the stenotic lesion 18. The method 110 further comprises fracturing in box 116 the stenotic lesion 18 in the body vessel on each radial plane of fracture with the balloon and the device 12.

FIGS. 9 through 11 illustrate an atherosclerosis cutting assembly 210 in accordance with another embodiment of the present invention. As shown, the assembly 210 includes similar components as in the assembly 10 depicted in FIGS. 1-3 and 7a and 7b. For example, the wire guide 54, outer catheter 60, and introducer sheath 56 of the assembly 10 in FIGS. 1-3 are similar to the wire guide 254, outer catheter 260, and introducer sheath 256 of the assembly 210 in FIGS. 9-11. However, in this embodiment, the distal and proximal rings 220, 224 of the cutting device 212 are each attached or integral with the expandable balloon 216 of the balloon catheter 214. The rings 220, 224 may be attached to the expandable balloon by any suitable means, e.g., by thermal bonding. In this embodiment, the cutting device 212 is pre-aligned about the expandable balloon 216 to further facilitate ease of placing both the cutting device 12 and the expandable balloon 216 at the lesion 218 of the body vessel 219.

While the present invention has been described in terms of preferred embodiments, it will be understood, of course, that the invention is not limited thereto since modifications may be made to those skilled in the art, particularly in light of the foregoing teachings.

1. An angioplasty cutting device for balloon angioplasty of a stenotic lesion in a body vessel, the device comprising:
a distal ring configured to be disposed at the distal end of the stenotic lesion relative to the device;
at least one strut attached to the distal ring and proximally extending therefrom, the at least one strut configured to be disposed at the stenotic lesion to engage the stenotic lesion for dilatation of the body vessel during angioplasty; and
a proximal ring configured to be disposed at the proximal end of the stenotic lesion relative to the device, the at least one strut being attached to the proximal ring and extending therefrom a predetermined length for delivery and retrieval of the device.

2. The device of claim 1 wherein the at least one strut is a plurality of struts to define a cutting body.

3. The device of claim 2 wherein each strut has a first portion and a second portion, each first portion being attached to the distal ring and extending longitudinally therefrom, the cutting body being radially expandable to engage the stenotic lesion for dilatation of the body vessel during angioplasty.

4. The device of claim 3 wherein the first portion is a distal portion and the second portion is a proximal portion of the at least one strut.

5. The device of claim 1 wherein the distal and proximal rings are made of polymeric material.

6. The device of claim 1 wherein the at least one strut is made of one of super-elastic material, shape memory material, metal, and polymeric material.

7. The device of claim 1 wherein the at least one strut extends proximally from the proximal ring for delivery and retrieval of the device.

8. The device of claim 1 wherein the at least one strut is attached to the distal ring by bonding.

9. The device of claim 8 wherein the at least one strut is bonded to the distal ring by sonic welding, thermal bonding, or adhesive bonding.

10. The device of claim 1 wherein the at least one strut is connected integrally with the distal ring and the proximal ring.

11. An atherosclerosis cutting device coaxially adaptable about an expandable balloon for angioplasty of a stenotic lesion in a body vessel, the device comprising:
a distal ring configured to be disposed adjacent the distal end of the balloon relative to the device;
a cutting body including a plurality of struts, each strut having a first portion and a second portion, each first portion being attached to the distal ring and extending longitudinally therefrom, the cutting body being radially expandable with the balloon to engage the stenotic lesion for dilatation of the body vessel during angioplasty; and
a proximal ring configured to be disposed adjacent the proximal end of the balloon relative to the device, each second portion being attached to the proximal ring, one of the struts extending therefrom a predetermined length for delivery and retrieval of the device.

12. The device of claim 11 wherein the distal and proximal rings are made of a polymeric material.

13. The device of claim 11 wherein the struts are made of one of shape memory material, super elastic material, metal, and polymeric material.

14. The device of claim 11 wherein the struts extend proximally from the proximal ring for delivery and retrieval of the device.

15. The device of claim 11 wherein each of the struts are radially attached to the distal ring by bonding.

16. The device of claim 15 wherein each of the struts are bonded to the distal ring by sonic molding, thermal bonding, or adhesive bonding.

17. The device of claim 11 wherein the struts are integrally connected to the distal and proximal rings.

18. An angioplasty cutting apparatus for treatment of a stenotic lesion in a body vessel, the device comprising:
a balloon catheter having a tubular body portion and an expandable balloon attached to and in fluid communication with the tubular body for angioplasty at the stenotic lesion, the expandable balloon having distal and proximal portions; and
an angioplasty cutting device coaxially adaptable about the expandable balloon for angioplasty of the stenotic lesion in the body vessel, the device comprising:
a distal ring configured to be disposed adjacent the distal end of the balloon relative to the device;
a cutting body including a plurality of struts, each strut having a first portion and a second portion, each first portion being attached to the distal ring and extending longitudinally therefrom, the cutting body being radially expandable with the balloon to engage the stenotic lesion for dilatation of the body vessel during angioplasty; and
a proximal ring configured to be disposed adjacent the proximal end of the balloon relative to the device, each second portion being attached to the proximal ring, one of the struts extending therefrom a predetermined length for delivery and retrieval of the device.

19. The apparatus of claim 18 wherein the struts are disposed longitudinally adjacent the distal end of the balloon relative to the device.

20. The apparatus of claim 18 wherein the struts extend proximally from the proximal ring for delivery and retrieval of the device.
